(19) United States
(12) Patent Application Publication
(10) Pub. No.: US 2009/0048655 A1
(43) Pub. Date: Feb. 19, 2009

(54) Two-step/dual-diameter balloon angioplasty catheter for bifurcation and side-branch vascular anatomy

(76) Inventor: G. David Jang, Redlands, CA (US)

Correspondence Address: LAURENCE H. PRETTY, 28th Floor, 633 West 5th Street, Los Angeles, CA, 90071, US

(21) Appl. No.: 12/229,991

(22) Filed: Aug. 27, 2008

Related U.S. Application Data
(63) Continuation of application No. 10/943,787, filed on Sep. 17, 2004.

(51) Int. Cl. A61F 2/84; A61M 25/10

(52) U.S. Cl. 623/1.11; 604/103.07

(57) ABSTRACT

The present invention tackles the challenging anatomic characteristics of the coronary artery disease in the bifurcation point and the origin of side-branch. The invention has a specifically designed angioplasty balloon catheter, particularly the balloon shape and profile, to be used in the diseased vessels at these difficult anatomic locations. In stent implanting into a coronary artery, a balloon catheter application is an inseparable requirement. A stent is a passive device that cannot be deployed in a diseased or stenosed artery without a pre-stent, with-stent and/or post-stent balloon dilatation. In majority (more than 95%) of available coronary stents, a stent is deployed by balloon expandable mode, meaning that the stent is delivered and expanded inside a vessel lumen by expanding a delivery balloon. This is done by crimping a stent over a folded balloon for delivery into a coronary artery. When expanded by balloon inflation, a stent is expanded and shaped passively by the inflated balloon shape and profile. The balloon catheter is designed to do angioplasty in the bifurcation and side-branch anatomy of coronary arteries, while minimizing the side effect. This specially designed balloon catheter is not only for balloon angioplasty dilatation of the bifurcation and side-branch anatomy, but is also for delivering and deploying specially designed bifurcation or side-branch stents into these difficult anatomic locations, as a stent delivery system.

### CROSS-REFERENCE TO RELATED APPLICATIONS

This application is a continuation of applicant's U.S. application Ser. No. 10/943,787 filed on Sep. 9, 2004, for which applicant claims the benefit of its filing date under 35 U.S.C. Sec. 120 and such application is hereby incorporated by reference. This application is also a related application to applicant's U.S. provisional application Ser. No. 60/499,990 filed Sep. 3, 2003 which is hereby incorporated by reference.

### BACKGROUND OF THE INVENTION

1. Field of Invention

This invention relates generally to percutaneous balloon coronary angioplasty (PTCA) and coronary stent delivery devices and methods, and more particular to PTCA and coronary stent delivery devices and methods suitable for bifurcation and side-branch anatomies

2. Description of the Related Art

By 2002, the percutaneous balloon angioplasty and stent implant procedures have become the dominant non-surgical revascularization method of the atherosclerotic stenosis, or obstruction, of the vascular lumen, and particularly in the coronary vascular system in the heart. With balloon angioplasty alone, without use of stent, the restenosis rate after angioplasty has been as high as 25-35% in the first time clinical cases. With use of bare stents in conjunction with balloon angioplasty, the restenosis was reduced significantly. Even so, the restenosis rate after stent implant is reported as 10-20% range depending on the condition of a vessel stented or what specific stent brand was used, requiring a need for further restenosis reducing measures after intravascular stenting.

To further reduce the restenosis rate after stent implant, numerous means designed to reduce restenosis rate has been tried, including laser, atherectomy, high frequency ultrasound, radiation device, local drug delivery, etc. Although the brachytherapy (radiation treatment) has proved to be reasonably effective in further reducing restenosis after stent implant, using brachytherapy is very cumbersome, inconvenient and costly. Mainly because it is a radioactive device with a declining isotope half-life, and radiation therapy specialist from another department has to be involved with the interventional cardiologist in the cardiac catheterization laboratory. The laser and atherectomy devices proved to be marginally useful in this purpose with added costs.

By 2003, drug coated, drug-eluting, stents have been introduced into the U.S. market after an FDA approval. The first U.S. approved drug-eluting stent has Sirolimus, an immune-suppressive drug, as main agent as anti-restenosis. This stent has further reduced a medium term restenosis down to 5-10% range. A cancer treatment drug; Paclitaxol, coated stent is in the clinical testing stage in mid 2003. Both of these drug-eluting stents has changed dramatically the restenosis rate after coronary stent implants.

With these promising restenosis rate improvements made with the drug-eluting stents, potential prospect for angioplasty and stent implant of bifurcation or side branch lesions of coronary anatomy has also improved. However, successful stent strategy for angioplasty and stenting of bifurcation or side-branch lesions requires two very fundamental elements. First is a specially designed stent that will readily adopt to a set of complex anatomic characteristics of a coronary artery lesion at a bifurcation or side-branch origin, which is far more complex and difficult for a stent to optimally adopt to. A stent that is designed for a regular vessel that is basically a single lumen tubular structure, can not adopt to a multi-lumen and multi-diameter bifurcation lesions. The next requirement is a specially designed angioplasty-stent delivery balloon catheter that is adoptable to the complex anatomic characteristics of a bifurcation or side-branch origin lesions. A specially designed stent cannot be effectively used if there is no specially designed angioplasty-stent delivery balloon catheter that is adopted to the anatomic characteristics of a bifurcation or side-branch origin lesions of coronary artery.

There is a need for an angioplasty-stent delivery balloon catheter that is adapted to the anatomic characteristics of a bifurcation or side-branch origin lesions of coronary artery. There is a further need for a specially designed angioplasty-stent delivery balloon catheter system for bifurcation or side-branch origin applications. There is yet a further need for a stent that is suited for bifurcation or side-branch lesions.

### SUMMARY OF THE INVENTION

Accordingly, an object of the present invention is to provide an improved angioplasty stent delivery balloon catheter.

Another object of the present invention is to provide an angioplasty stent delivery balloon catheter adapted to the anatomic characteristics of a bifurcation or side-branch origin lesions of coronary artery.

A further object of the present invention is to provide an angioplasty stent delivery balloon catheter, and stent, that are adapted to the anatomic characteristics of a bifurcation or side-branch origin lesions of coronary artery.

These and other objects of the present invention are achieved in a balloon catheter for use in a vascular bifurcation or side-branch anatomy. A catheter body is provided. A balloon is positioned at a distal portion of the catheter body. The balloon has a balloon outer skin, a first lumen adapted to receive a guidewire and a second lumen configured to provide inflation and deflation of the balloon. The balloon has a first section with a first average diameter, and second section with a second average diameter that is smaller than the first average diameter. The first and second sections are coupled by a transition section that has a geometry and is sized to reduce vessel damage when positioned at a point of vessel bifurcation.

In another embodiment of the present invention, an angioplasty balloon catheter is provided for use in a vascular anatomy and includes an angioplasty catheter body. A tubular balloon is coupled to a distal end of the angioplasty catheter body. The tubular balloon includes a shaped balloon skin, a catheter shaft with a first lumen configured to receive a guidewire and a second lumen configured to be provide inflation-deflation of the balloon. The balloon has a shaped outer geometry and is size to reduce vessel damage when positioned at a point of vessel bifurcation.

In another embodiment of the present invention, a stent delivery device includes a catheter body. A balloon is positioned at a distal portion of the catheter body. The balloon includes a balloon outer skin, a first lumen adapted to receive a guidewire, and a second lumen configured to provide inflation and deflation of the balloon. The balloon has a first section with a first average diameter, and a second section with a second average diameter that is smaller than the first average diameter. The first and second sections are coupled by a transition section that has a geometry and is sized to reduce vessel damage when positioned at a point of vessel bifurcation. A vascular stent is positioned on an exterior of the balloon exterior.

In another embodiment of the present invention, a method of treating a vascular bifurcation or side-branch anatomy provides a catheter that includes a balloon with a transition section that couples a first section with a second section. The transition section has a geometry and size configured to reduce vessel damage when positioned at a point of vessel bifurcation. A stent is mounted in a non-expanded state on an exterior of the balloon. The catheter with the stent in a non-expanded state is positioned at a vascular bifurcation or a vascular side-branch site. The balloon is inflated. The stent is deployed in an expanded state at the vascular bifurcation or vascular side-branch site. The catheter is removed from the vascular bifurcation or a vascular side-branch site.

### BRIEF DESCRIPTION OF DRAWINGS

FIG. 1 is a side view of a balloon catheter of the present invention in an inflated state illustrating the first and second sections with different balloon diameters, coupled together by a transition section and including a balloon marker.

FIG. 2 is a longitudinal cross-sectional view of the FIG. 1 balloon catheter.

FIG. 3(a) is the FIG. 2 balloon catheter with an outer-mounted, expanded stent shaped by the inflated shape of the balloon.

FIG. 3(b) is a longitudinal cross-sectional view of the FIG. 3(a) expanded stent, of the present invention, with the balloon assembly removed from the stent lumen.

FIG. 4 is a side view of one embodiment of a balloon catheter of the present invention with a deflated and folded balloon illustrating a transition section and dissimilar proximal and distal folded balloon profiles.

FIG. 5 is a side view of the FIG. 4 balloon catheter with a stent crimp-mounted over the folded balloon for delivery and deployment.

FIG. 6 illustrates an over-the-wire embodiment of the FIG. 1 balloon catheter.

FIG. 7 illustrates a rapid-exchange embodiment of the FIG. 1 balloon catheter.

### DETAILED DESCRIPTION

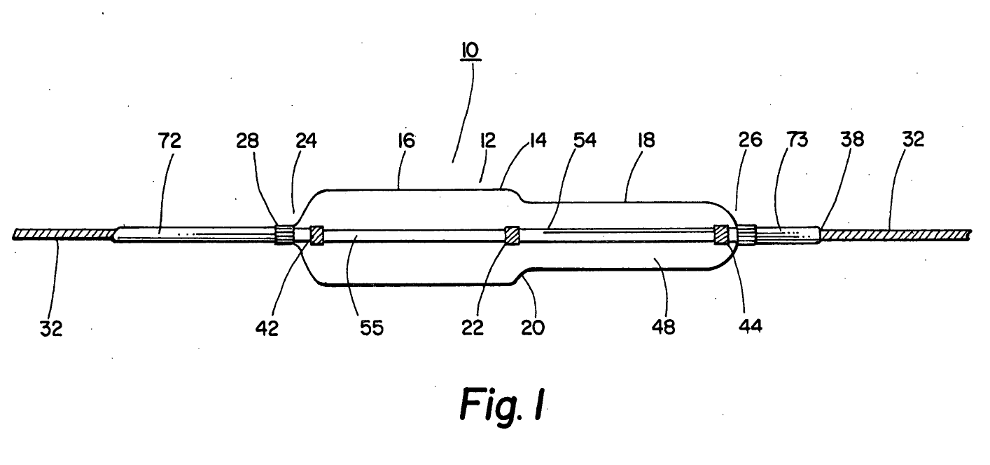

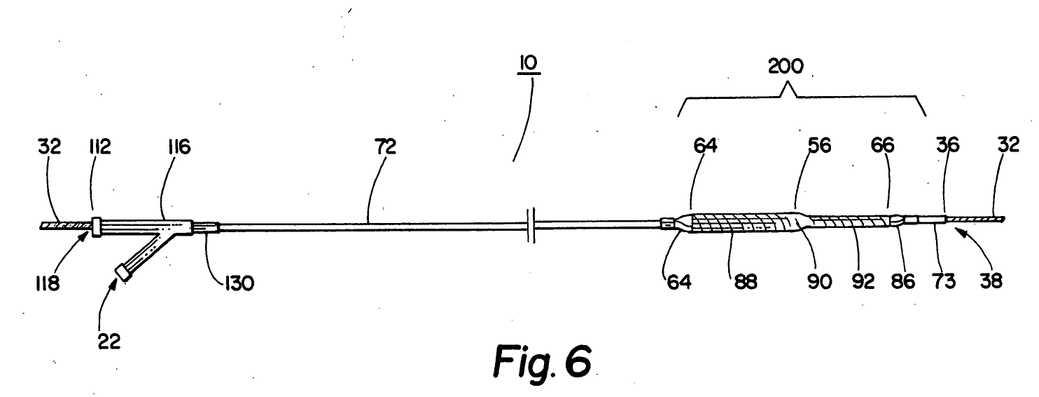

Referring to FIG. 1, one embodiment of a balloon catheter 10 according to the present invention will now be described. The balloon catheter 10 includes a balloon 12 positioned at a distal portion of catheter shaft 72 (see FIG. 6). The balloon 12 has a balloon outer skin 14. In this embodiment, balloon 12 may have a first section 16 with a first average diameter and second section 18 with a second average diameter that is smaller than first average diameter. First and second sections 16 and 18 are coupled by a transition section 20 that has a geometry and is sized to reduce vessel damage when positioned at a point of vessel bifurcation.

Balloon catheter 10 is particularly suited for use in stenting bifurcation or side-branch origin lesions. Balloon catheter 10 is configured to provide proper and/or successfully implantation of a stent at bifurcation or side-branching origin lesions. Coronary bifurcations have variable sets of complex anatomic characteristics that are met with the use of balloon catheter 10 with first section 16, second section 18 and transition section 20. Balloon catheter 10 is configured to carry a stent, in a non-expanded state, and deliver the stent to bifurcation or side-branching origin lesions. Balloon 12 is then expanded and molded into an elongated tabular structure by its external shape when inflated with pressurization by a variety of means including but not limited to the introduction of a fluid such as saline and the like.

In one specific embodiment, a stent is expanded and deployed with a nominal inflating pressure of about 8-10 ATM (atmospheric pressure) that is exerted by balloon 12. In another embodiment, balloon 12 can be expanded in a pressure of 20 ATM or more.

Transition section 20 can have a proximal-to-distal step-down between first and sections 16 and 18. Balloon 12 can include a radiopaque marker 22 to coincide with transition section 20. Radiopaque marker 22 can be positioned at a number of different locations, including but not limited to, proximal, distal and intermediate positions of transition section 20.

Balloon catheter 10 can be utilized as both a balloon angioplasty and a stent delivery system for bifurcation and side-branch origin anatomies of coronary vessels. Balloon catheter 10 can be a modular system, as described hereafter. In a stent implant procedure, particularly in complex anatomic environment like in bifurcation lesions, a pre-stent balloon dilatation of the stenotic lesion is often a pre-requisite. Balloon catheter 10 can be used as a balloon angioplasty device alone, as a pre-stent pre-dilatation device, as a stent delivery tool, and the like.

In a bifurcation anatomy, if only one side-branch and its origin has a stenotic lesion, balloon 12 is inserted in the side-branch with a distal small diameter segment 18, and the large main branch with a proximal large diameter segment 16. Radiopaque marker 22 is used as a guide to position transition section 20 at the bifurcation point under fluoroscopy for either angioplasty or stent delivery purposes. In each procedure, transition section 20 may be placed at the side-branch origin using radiopaque marker 22, which can coincide with the location of transition section 20. With balloon 12, if radiopaque marker 22 is properly positioned at the side-branch origin (i.e., at bifurcation point), the distal small diameter segment 18 and proximal large diameter segment 16 of the balloon tube are properly placed, respectively, in the smaller side-branch and the larger main branch. Similarly, when balloon 12 is used as a stent delivery system for a bifurcation stent, radiopaque marker 22 is the key guide under fluoroscopy to position transition section 20 of a stent 56 at the bifurcation point.

Stent 56 can be a passive device that is not self expanding. When balloon 12 is inflated, stent 56 is expanded and molded in positioned at the bifurcation anatomy with first section 60 in the main branch, second section 62 in the side-branch and transition section 58 at the bifurcation point (i.e., side-branch origin). Once stent 56 is deployed and expanded, a jail-break balloon dilatation on the stent wall that blocks the distal main branch beyond the side-branch origin is desired. In one embodiment of the present invention, a size of a jail-broken stent cell should match the size and diameter of the vessel distal to the side-branch origin. For this purpose of optimally jail-broken cell size, stent 56 that is specifically designed for bifurcation application can have a properly planned reserve cell boundary for a sufficient stretching into an optimal jail-broken cell size.

If all three vessel segments of a bifurcation anatomy are affected by an atherosclerotic lesion, (i.e., a proximal main branch and two distal side branches), all three vessel segments of the bifurcation may need angioplasty and stenting. Balloon 12, as part of a modular system, is effective for this anatomy. First and second sections 16 and 18 can deliver two separate stents at the bifurcation lesion. A first set of balloon 12 delivers and deploys a stent 56 into the first side branch. A proximal larger diameter segment 60 of stent 56 is deployed in the proximal main branch. A distal smaller diameter segment 62 of stent 56 is deployed in the first side-branch, simultaneously.

After jail-breaking the side-wall of proximal larger segment 60 of stent 56 to open the blocking struts to the distal un-stented branch, a second set of balloon 12 delivers and deploys a stent 56 into the second side branch, repeating the similar procedural steps as the first side-branch stenting. When the second stent is deployed, the main branch proximal to the bifurcation or side-branch point has two over-lapping stent segments 60. At this point, the side-wall of the proximal larger segment 60 of the second stent struts blocks the orifice of the first side-branch which already was stented. This requires another, second, jail-breaking of the side-wall of the proximal larger segment 60 of the second stent to open the orifice of the first side-branch that received the first stent.

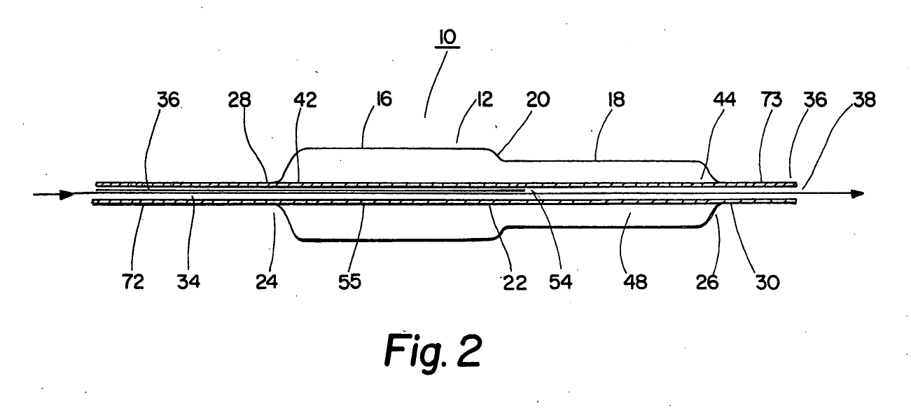

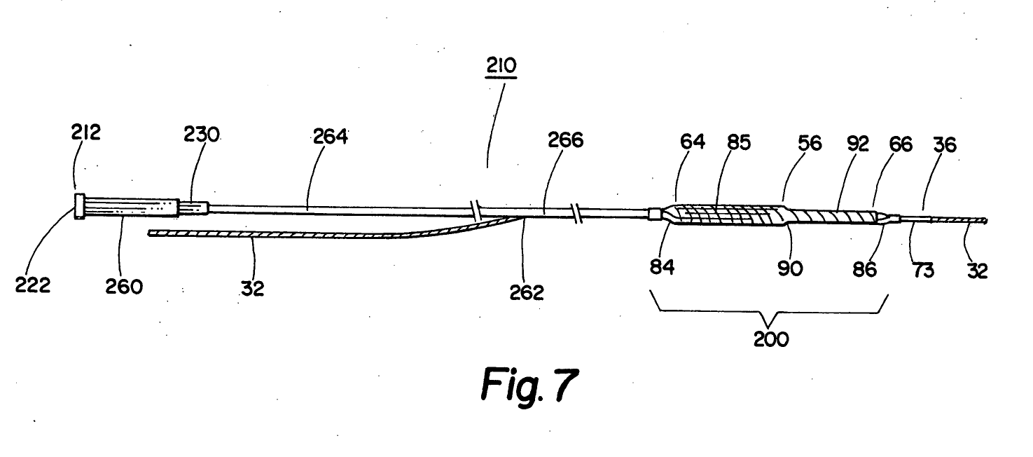

Balloon 12 can be made both in an over-the-wire exchange system illustrated in FIG. 6, and in a rapid-exchange system, as illustrated in FIG. 7. Balloon catheter 10 of both a rapid-exchange system and an over-the-wire system can be identical. Balloon catheter 10 is shown with balloon 12 in an inflated side view in FIG. 1, a longitudinal cross-sectional view in FIG. 2, a folded-balloon view and a stent 56-mounted view over a balloon 12 in a folded configuration 70.

The profile and configuration of balloon 12 is illustrated in an inflated state in FIG. 1. Proximal and distal ends 24 and 26 of balloon 12, respectively, are on a catheter shaft at positions 28 and 30 can be achieved according to well known balloon catheter fabrication methods. A guidewire 32 is in place in a guidewire lumen 34 (see FIG. 2) that runs through a longitudinal axis of a shaft of balloon catheter 10.

Also illustrated are a distal tip 36 of balloon catheter 10 and a distal port 38 of guidewire lumen 34. Balloon 12 has first and second sections 16 and 18 that are coupled with a transition section 20. Balloon 12 is made of balloon skin 14 and maintains an enclosed balloon lumen 40. Balloon skin 14 can be made of a variety of different materials, including but not limited to, polyethylene, nylon, PET, other polymer combinations, and the like. It will appreciated that balloon 12 can be made of any suitable material used in fabricating balloon skin 14. In the FIG. 1 embodiment, three markers 22, 42, and 44 are provided, and balloon 12 has an inflation-deflation lumen opening 46.

Balloon 12 has a dual-diameter balloon silhouette, denoted as first and second sections 16 and 18. Transition section 20 has a geometry and is sized to reduce vessel damage when positioned at a point of vessel bifurcation. Generally, first section 16 has an average diameter that is larger than an average diameter of second section 18. First and second sections 16 and 18 can have lengths that are about the same or different, with first section 16 being longer or shorter than second section 18.

In the embodiment illustrated in FIG. 1, the longitudinal margins of first section 16 of balloon skin 14 are roughly parallel to each other, and diameter is substantially the same along the length of first section 16. In another embodiment, all or a portion of the longitudinal margins can be non-parallel, such as in a tapered configuration, and at least a portion of the diameters along the length of first section 16 are different. This is the case when first section 16 has a tapered geometry. Similarly, the longitudinal margins of second section 18 can also be roughly parallel to each other as well as non-parallel, such as in a tapered configuration. At least a portion of the diameters of second section 18 can be different.

Balloon catheter 10 is particularly useful for vascular bifurcation or side-branch anatomies. Balloon catheter 10 may be a balloon angioplasty and stent delivery catheter configured for use in specific anatomic characteristics of a bifurcation or side-branch origin lesions of coronary artery. A bifurcation in coronary anatomy is created when a main branch gives rise to a side branch. A side-branching of coronary anatomy results in a hub that is connected to three separate segments of branches: a main branch proximal to the branching point, a new side branch distal to the branching point and an extension of the main branch distal to the branching point. In this situation, the branching point becomes a bifurcation. In other words, a bifurcation is formed when an artery divides into two distal branches.

Regarding the side-branch vs. bifurcation anatomic definition, a bifurcation means a dividing point where one coronary artery branch becomes into two branches. Therefore, any side-branch take-off point is technically interchangeable with a bifurcation point. In a practical sense, a side-branch point is a bifurcation point and a bifurcation point is a take-off point of a side-branch. One unique instance is where a main branch divides into two equal sized caliber branches. In this instance, either one of the two bifurcated branches could be called the main branch anymore. Or both could be called bifurcated side-branches. Inmost of these instances of bifurcation vessel anatomy, the main branch before a side-branch take-off, or before two equally bifurcated branches, remains a larger diameter vessel and a side-branch or equally bifurcated branches become smaller caliber vessel(s). For practical purpose, a side-branching and bifurcation can be termed interchangeably in most of the situation, except perhaps in few exceptions. In this disclosure and discussions, bifurcation point and side-branch point is used concurrently or interchangeably.

The anatomy of a bifurcation can have three different vessel diameters. There can be at least be two different vessel diameters associated with a bifurcation point. When an atherosclerotic lesion develops at a bifurcation, one, two or all three branches can be involved with atherosclerotic plaques. Furthermore, an angle at which a side branch takes off from the main branch also has a wide range of variations. %

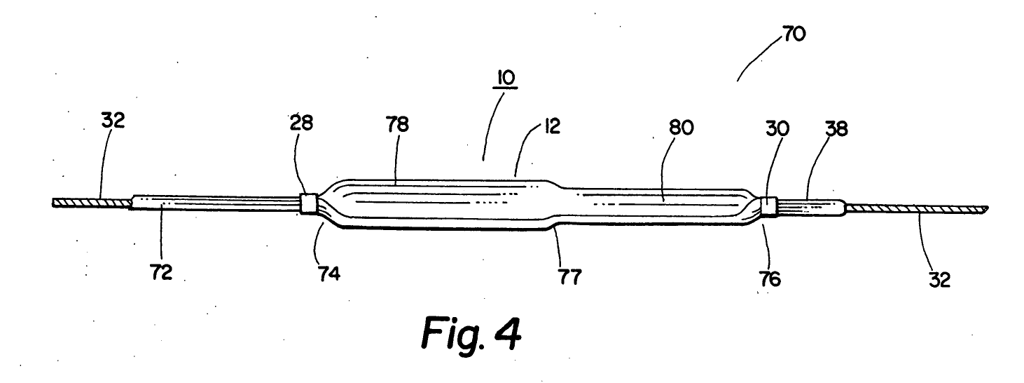

A side-branch arises from the main branch at varying angles of take-off. Balloon catheter 10 is delivered to a side-branch take-off point in a folded delivery mode. Second section 18 enters into the side-branch, while first section 16 stays in the main branch. Balloon 12 dilates both the proximal and distal zones of the side-branch take-off point at the same time. When a folded balloon in delivery mode 60 (as seen in FIG. 4) enters a side-branch, balloon 12 and its shaft bend at an angle to accommodate the angle of take-off of the side-branch from the main branch. A degree of bending of balloon 12 in delivery mode 60 can be determined by a degree of the take-off angle of the side-branch.

At an insertion stage of balloon catheter 10 for a angioplasty or stenting procedure, placement of balloon 12 in the coronary side-branch point causes a bending of balloon 12 along with the catheter shaft. The exact point of bending of balloon 12 is preferable at transitional section 20 and coincides with the transitional point between the larger diameter proximal branch and the smaller diameter side-branch of coronary anatomy. When balloon 12 is inflated in place, first section 16 stays in the proximal larger caliber main coronary branch, and second section 18 occupies the space in the distal small caliber side-branch. If first section 16 is prolapsed into the smaller caliber side-branch, the small caliber side-branch can have an intimal tear or dissection as a complication. Conversely, if second section 18 is prolapsed into the proximal large diameter main artery, second section 18 can be a cause for a possible serious problems. Proper placement of balloon 12 in the side-branch or bifurcation is critical not only for balloon dilatation but also for stent placement when balloon catheter 10 is used as a stent delivery and deployment vehicle.

One or more radiopaque markers can be included with balloon catheter 10 to provide for a more precision placement of balloon 12 in a side-branch or bifurcation point. In the Figure-i embodiment, three balloon markers 22, 42, and 44 are provided. One marker 22 is in the middle, another one 42 near or at proximal end 24, and another one 44 to mark distal end 26 of balloon 12.

In one embodiment, radiopaque marker 22 is placed in transition section 20. In one embodiment, a radiopaque marker 22 is a middle marker designed to indicate the location of the transition section 20 between first section 16 and second section 18. Middle marker 22 is positioned at transition section 20, or in sufficiently close proximity, as to enable the operator to position, under fluoroscopy, transition section 20 at the side-branch take-off or bifurcation point in coronary artery during an angioplasty or stenting procedure. Once middle marker 22 and transition section 20 are accurately placed at the bifurcation point of the coronary anatomy, balloon catheter 10 is ready for dilatation at the exact desired location. Radiopaque marker 22 can be circumferentially attached on a catheter shaft 55 inside balloon lumen 48 to accurately indicate the location of transition 20. Middle marker 22 can be placed near, but not exactly, at the location of transition section 20 and can still accurately indicate the position of transition section 20 under fluoroscopy during an angioplasty procedure.

A stent 56 is provided that is particularly designed for a bifurcation or side-branch application. In this embodiment, stent 56 has a transition section 58 that is positioned between a first section 60 and a second section 62. First section 60 has a larger average diameter than an average diameter of section 62. When crimp-mounting stent 56 for delivery on balloon 12 of the present invention, transition section 58 of stent 56 should also be placed to coincide with middle marker 22 so that stent 56 is deployed accurately at a bifurcation or side-branch by using the reference of middle marker 22 under fluoroscopy during a procedure. Stent 56 is then correctly molded and deployed in the bifurcation or side-branch by dilating the balloon 12 with first and second sections 16 and 18 in the vessel lumen.

A central shaft of balloon 12 can carry middle marker 22 and inflation-deflation lumen opening 54. As previously discussed, middle marker 22 indicates the location of transition section 20 but is not necessary located at a position that indicates the center of balloon 12. A length ratio between first section 16 and second section 18 may be variably changed as necessary. Therefore, the position of transition section 34 may also be variably shifted along the longitudinal length of balloon 12. In one embodiment, middle marker 22 is designed to follow the location of transition section 34 which may shift up or down the longitudinal axis of balloon 12, and need not necessarily indicate the center of balloon shaft 44.

The location of inflation-deflation lumen opening 54 can be placed at almost any location inside balloon lumen 38. Many single lumen balloons have an opening in the proximal end of the balloon lumen. In the embodiment illustrated in Figure-i, inflation-deflation lumen opening 54 is placed distal to middle marker 22 and distal to transition section 34. This particular configuration has a purpose.

When balloon 12 is inflated in a bifurcation or side-branch take-off point, balloon skin 14 may slide proximally toward the larger diameter side of the vessel anatomy. Inflation-deflation lumen opening 54 inflates second section 18 earlier than first section 16 when inflation-deflation lumen opening 54 is second section 18. Thus, second section 18 is inflated first and anchors distal end 26 of balloon 12 to prevent sliding of balloon skin 14 proximally into the large caliber main branch of the coronary artery. This may be more significant when balloon catheter 10 is used for stent delivery to a bifurcation or side-branch take-off point.

By way of illustration, stent 56 can slide forward or backward during a stent deployment phase if the vessel anatomy is in a certain condition, such as the one described above. Because bifurcation or side-branch stenting involves two dissimilar caliber vessels within the length of stent 56, as discussed in the earlier paragraphs, sliding of stent 56 during deployment can cause undesirable consequences. By inflating second section 18 first and placing inflation-deflation lumen opening 54 in the distal zone, sliding of stent 56 during deployment in a bifurcation or side-branch anatomy may be prevented.

FIG. 2 illustrates balloon catheter of FIG. 1 in a longitudinal cross-section diagram. In the FIG. 2 embodiment, three markers 22, 42, 44, guidewire 32 and inflation deflation lumen 54 are shown. Guidewire 32 traverses through guidewire lumen 34. Balloon 12 is bonded on a catheter shaft at positions 18 and 20. Distal port 20 of guidewire 32 is also shown.

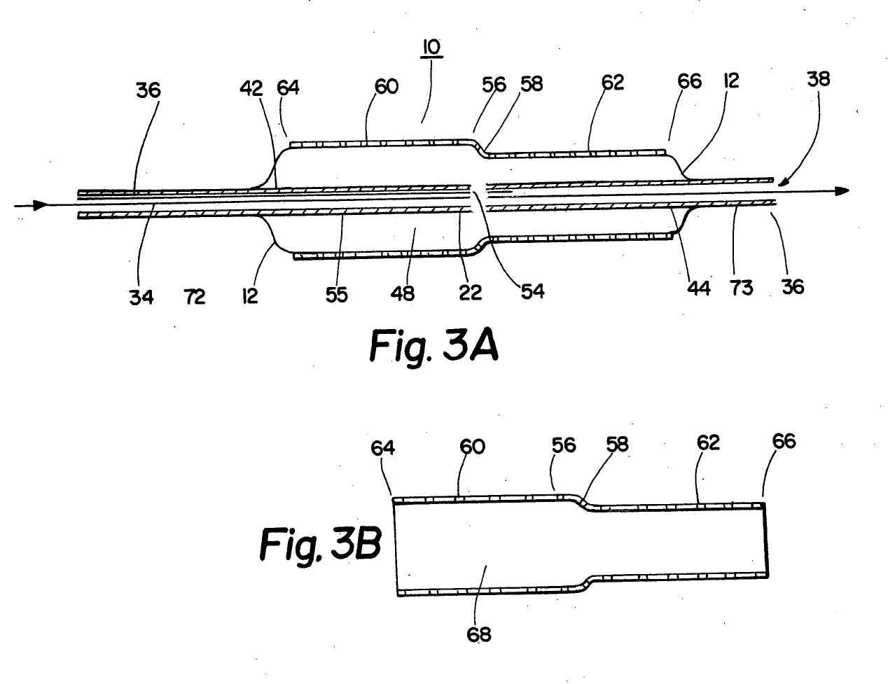

Referring now to FIG. 3(a), the same longitudinal cross-sectional view of balloon catheter 10, from FIG. 2, now includes an expanded two-step stent 56 that is in a surrounding position around the balloon 12 which is an inflated, two-step dilation balloon. As indicated earlier, a stent is a very passive device that is generally expanded by balloon inflation. In one embodiment, a self-expanding stent is not used with balloon catheter 10 of the present invention. In FIG. 3(a), stent 56 is passively shaped, forms generally to the geometry of balloon 12 and follows the two-step pattern of sections 16 and 18. Stent 56, in an expanded state, has a proximal end 64 and a distal end 66. In FIG. 3(a), stent 56 has a first section 60 with a larger average diameter than an average diameter of a second section 62. First and second sections 60 and 62 are joined at a transition section 58, where a proximal-to-distal step down of a diameter transition of stent 56 occurs.

First section 60, second section 62 and transition section 58 of stent 56 are correlated in geometry and size to first section 16, second section 18 and transition section 20 of balloon 12. Transition section 58 of stent 56, transition section 20 of balloon 12 and middle marker 22 of balloon 12 are also correlated. During an angioplasty or stent procedure, middle marker 22 is the guide for the operator to place middle marker 22 at the precise location of a side-branch or bifurcation of coronary artery. In various embodiments, balloon 12 is a two-step angioplasty balloon suitable to perform angioplasty in side-branch lesions of coronary artery, and also as a bifurcation stent delivery system. The two-step/two-diameter configuration of balloon 12 is suitable for delivery and shaping of stent 56 in bifurcation lesions that includes proximal large and distal small caliber vessel branches.

Referring now to FIG. 3(b), stent 56, which has the same two-step/dual diameter configuration, is illustrated as being expanded and shaped by balloon 12, and balloon 12 has been removed. In FIG. 3(b) stent 56 is essentially the same stent as that of FIG. 3(a). Stent 56 has the same proximal end 64 and distal end 66, along with an expanded lumen 68. Transition section 58 borders first section 60 and the smaller second section 62. The shape of expanded stent 56 is a critical element of the design of stent 56 for bifurcation use. Stent 56 is deployed and molded in place in a bifurcation lesion by a stent delivery system that utilizes balloon 12 with the two-step/dual-diameter geometry. Balloon 12 has a geometry that is configured to provide deployment and molding of the shape of stent 56 for its deployment in a bifurcation or side-branch lesion of coronary artery.

FIG. 4 illustrates a side view of balloon catheter 10 with balloon 12 folded into a low profile shape 70 for angioplasty, or for crimping a stent over the folded balloon 12 for delivery. Balloon catheter 10 has a proximal shaft 72, a distal shaft portion 73, distal tip 38 and guidewire 32 positioned in guidewire lumen 34 and runs through the entire length of the catheter shaft. Balloon 12, in the folded state illustrated in FIG. 4, extends from proximal end 74 and distal end 76. In the FIG. 4 embodiment, first section 78 has a larger average diameter than that of second section 80, as well as a larger average diameter than that of transition section 58.

With balloon 12 in the FIG. 4 folded configuration, it is now ready for a balloon dilatation angioplasty. Balloon catheter 10 can be utilized with, classic balloon angioplasty, pre-dilatation angioplasty for stent implant, post-dilation after a stent implant, and the like. As illustrated in FIG. 4, balloon 12 is suitable for bifurcation and side-branch anatomies and is shown as being in a folded configuration with a low profile state for a balloon angioplasty application.

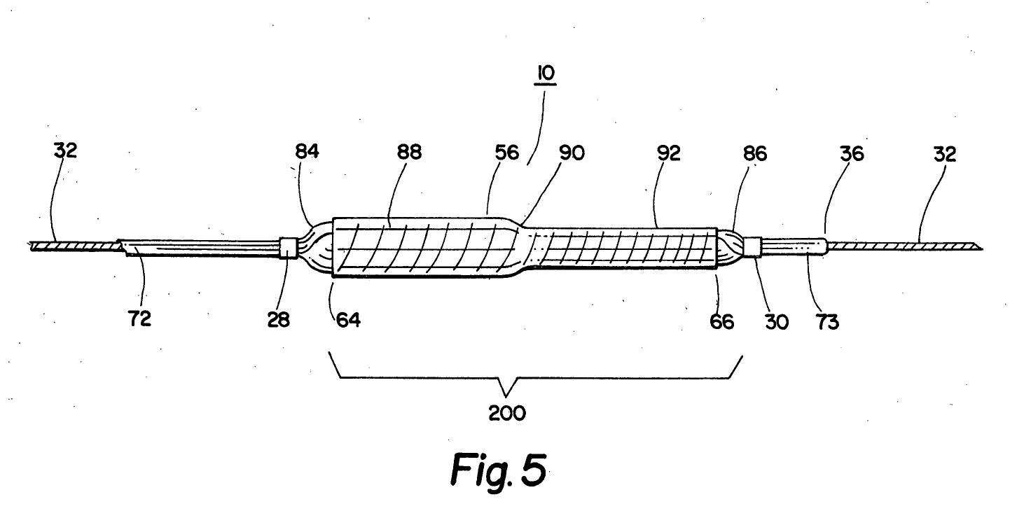

Similarly, balloon 12, in the folded state, is utilized for the delivery of stent 56. Stent 56 is crimped over the exterior of folded balloon 12. When stent 56 is mounted over folded balloon 12, balloon catheter 10 is ready for a bifurcation stent delivery as shown in FIG. 5.

In FIG. 5, stent 56 may be crimp-mounted over the FIG. 4 folded balloon 12. In this embodiment, stent 56 is shown in a state that readies it for delivery to a bifurcation or side-branch lesion in a coronary artery. In this embodiment, stent 56 is shown with a proximal end 64 and a distal end 66, and folded balloon 12 has an exposed proximal end 84 and an exposed distal end 86 that is not covered by stent 56. In this mounted state, stent 56 has a first section segment 88 coupled by a transition section 90 to a second section 92. The average diameter of first section 88 is larger then transition section 90 and second section 92, and the average diameter of transition section 90 is larger than an average diameter of second section 92. Stent transition section 90, transition section 77 of folded balloon 12 and radiopaque middle marker 22 underneath are aligned to coincide with each other. By positioning radiopaque marker 22 at a bifurcation or side-branch lesion, under fluoroscopy during a stenting procedure, both folded balloon 12 and stent 56 are automatically and correctly positioned in the bifurcation or side-branch lesion. Stent 56 is then expanded by when folder balloon 12 is inflated at the bifurcation or side-branch lesion. Stent 56 is transformed into the expanded two-step/dual-diameter stent 56 of FIG. 4.

In a real life implementation for a bifurcation or side-branch anatomy, crimp-mounted stent 56 and balloon 12 bend a certain way to conform to the coronary vessel anatomy. In an expanded state, stent 56 also conforms to the coronary vessel anatomy in a certain way, depending on the degree of conformability of the design of stent 56.

Stent 56 cannot be expanded and molded into a two-step/dual-diameter stent in a bifurcation or side-branch origin lesion unless it is delivered by balloon 12. In order words, stent 56 must be expanded by a balloon with has mutli-step/multi diameter geometry. In one specific embodiment, the folded balloon 12 has a first section 78, second section 80 and transition section 77. The balloon that deploys stent 56, e.g., balloon 12 is a two-step/dual diameter balloon.

FIG. 6 illustrates an embodiment with balloon catheter 10 is shown in an over-the-wire catheter exchange system. As seen in FIG. 6, balloon catheter 10 has a first lumen 118 adapted to receive a guidewire 32 and a second lumen 120 configured to provide inflation and deflation of balloon 12. In this embodiment, balloon catheter 10 has a proximal end 112 and a proximal guidewire lumen opening 118 on the left end of FIG. 6, and distal end 36 and a distal guidewire lumen opening 38 on the right end of FIG. 6. The proximal end of catheter shaft 72 has a Y-connector 116 with a guidewire lumen opening 118 and an inflation deflation lumen opening 122 with a connection distally to a strain-relief sleeve 130. Main shaft 72 of catheter 10 encompasses the entire length of catheter 10 from a proximally located Y-connector 116 and traverse through stent delivery balloon assembly 200 and ends in distal shaft 20. A guidewire 32 is positioned in place through guidewire lumen 34 of catheter 10. Balloon assembly 200 of balloon catheter 10 has the main business role as a bifurcation or side-branch angioplasty and stent delivery system. Balloon assembly 200 has basically similar configuration as illustrated in FIG. 5.

As illustrated in FIG. 7, a two-step/dual-diameter balloon tube assembly 200 is adapted for an angioplasty rapid-exchange catheter system. The distal end segment of the balloon catheter 210, including balloon assembly 200 and crimp-mounted stent 56, is exactly the same as FIG. 6. The catheter 210 has a proximal end 212 is made of a with an opening 222. The difference is in proximal end connector 260, a rapid-exchange proximal guidewire opening 262 and a hard metallic proximal shaft 264 of balloon catheter 10. Because proximal guidewire opening 262 is on the side of catheter shaft 266, moved to the distal catheter shaft proximal to balloon assembly 200, proximal end 212 is made of a simple tubular connector hub 260, providing an opening 222 for inflation-deflation of distally mounted balloon assembly 200. A guidewire 32 is entered into guidewire lumen 34 through proximal opening 262, which is an open orifice made on the side of a soft catheter shaft 266, and exits through distal guidewire opening 38 at distal end 26 of balloon catheter 210. As indicated above balloon assembly 200 at the distal end of balloon catheter 210 is same as FIG. 6, including the same two-step/dual-diameter balloon tube 12 and the middle radiopaque marker 22. The exactly same balloon assembly 200 of FIG. 6 is adapted to a rapid-exchange catheter system as illustrated in FIG. 7.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, the relative diameters of the balloon may be sized as shown in the figures. In one embodiment, the diameter of the larger section of the balloon greater than the diameter of the smaller section. Although not limited to the following, in other embodiments, the average diameter of the larger section of the balloon is about 5%, 10%, 15%, 20%, 25%, 30%, 40%, 50%, 60%, 70%, 80%, 90%, 100%, or other percentages greater than the average diameter of the smaller section of the balloon. Other details can be found in my provisional application U.S. Ser. No. 60/499,990 filed Sep. 3, 2003, which application is fully incorporated herein by reference.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. A shaped angioplasty balloon catheter for use at a transition point of a vessel at which the vessel has a main branch and a side branch of relatively larger and smaller diameters, respectively, and the transition point between them, the catheter comprising:
a catheter body including a catheter shaft having a first lumen, which is adapted to receive a guide wire, and a second lumen configured to provide an inflation and deflation path for inflation fluid,
a dual-diameter single balloon positioned at a distal portion of the catheter body inflatable from deflated condition to a relatively larger diameter inflated condition by fluid pressure applied through said second lumen, the dual-diameter balloon having an outer skin profile of two dissimilar diameters when inflated, said dual-diameter balloon having a first proximal section with a first average inflated diameter, and a second distal section with a second average inflated diameter positioned toward the distal end of said catheter body, the second average inflated diameter of said dual-diameter balloon being smaller than the first average inflated diameter, the first proximal and second distal sections of said dual-diameter balloon being joined by a balloon transition section, the first and second diameters and transition section of said balloon in their inflated condition being sized and shaped to reduce vessel damage when positioned at the transition point of the vessel,
at least one radiopaque marker positioned on said catheter shaft in alignment with the transition section of said balloon, said radiopaque marker being positioned during an angioplasty procedure at the vessel transition point under fluoroscopy prior to balloon inflation;
whereby said balloon, in its inflated condition, is positioned such that the first proximal section occupies the main branch, the second distal section occupies the side branch, and the transition section is positioned at the transition point of the vessel.

2. A stent delivery system for installing a shaped in situ dual diameter stent in a vessel which has a main branch and a side branch of relatively larger and smaller diameters, respectively, and a transition point between them, the stent delivery system comprising:
a catheter body including a catheter shaft having a first lumen, which is adapted to receive a guide wire, and a second lumen configured to provide an inflation and deflation path for inflation fluid, and
a dual-diameter single balloon positioned at a distal portion of the catheter body inflatable from deflated condition to a relatively larger diameter inflated condition by fluid pressure applied through said second lumen, the dual-diameter balloon having an outer skin profile of two dissimilar diameters when inflated, said dual-diameter balloon having a first proximal section with a first average inflated diameter, and a second distal section with a second average inflated diameter positioned toward the distal end of said catheter body, the second average inflated diameter of said dual-diameter balloon being smaller than the first average inflated diameter, the first proximal and second distal sections of said dual-diameter balloon being joined by a balloon transition section, the first and second diameters and transition section of said balloon in their inflated condition being sized and shaped to to be positioned within the main branch, side branch and transition point, respectively
at least one radiopaque marker positioned on said catheter shaft in alignment with the transition section of said balloon, said radiopaque marker being positioned at the vessel transition point under fluoroscopy during an angioplasty procedure prior to balloon inflation; and
an expandable stent closely fitted over and carried by said balloon in its deflated condition, inflation of said balloon to its inflated condition after said radiopaque marker has been positioned at the vessel transition point molding said stent in situ into a dual diameter shape positioned in the main branch, side branch and transition point of the vessel.

3. A method of treating a vascular vessel having a transition point at which the vessel has a main branch and a side branch of relatively larger and smaller diameters, respectively, and the transition point between them, the method comprising the steps of:
providing a catheter which includes: a catheter body having a catheter shaft with a first lumen, which is adapted to receive a guide wire, and a second lumen configured to provide an inflation and deflation path for inflation fluid; a dual-diameter single balloon positioned at a distal portion of the catheter body inflatable from a deflated condition to a relatively larger diameter inflated condition by fluid pressure applied through the second lumen, the dual-diameter balloon having an outer skin profile of two dissimilar diameters when inflated, said dual-diameter balloon having a first proximal section with a first average inflated diameter, and the second distal section with a second average inflated diameter positioned toward the distal end of said catheter body, the second average inflated diameter of said dual-diameter balloon being smaller than the first average inflated diameter, the first proximal and second distal sections of said dual-diameter balloon being joined by a balloon transition section, said first proximal, second distal and transition sections of the balloon being sized and shaped in their inflated condition to reduce vessel damage when positioned at the transition point of the vessel; and at least one radiopaque marker positioned on said catheter shaft aligned with said balloon transition section;
mounting a cardiovascular stent in overlapping contact with said balloon in its deflated condition:
advancing said catheter and said stent through the vessel in an angioplasty procedure under fluoroscopy until the radiopaque marker is positioned at the transition point of the vessel;
applying inflation pressure through the second lumen to inflate the balloon thereby causing the first proximal, second distal and transition sections of said balloon to inflate within the interior of the main branch, side branch and transition point, respectively of the vessel;
whereby the step of applying inflation pressure through the second lumen to inflate the balloon molds the stent in situ into a dual diameter shape sized to reduce vessel damage when the transition section of the balloon is positioned at the transition point junction of the vessel.
